(19) United States
(12) Patent Application Publication
(10) Pub. No.: US 2004/0186455 A1
(43) Pub. Date: Sep. 23, 2004

(54) Undergarment for incontinent and semi-incontinent male

(76) Inventors: Evelyn Menasha, Suffolk, VA (US); Cecilia Weisberger, Virginia Beach, VA (US)

Correspondence Address: BRADLEY D. GOLDIZEN, 505 SOUTH INDEPENDENCE BOULEVARD, SUITE 102, VIRGINIA BEACH, VA, 23452, US

(21) Appl. No.: 10/390,858

(22) Filed: Mar. 18, 2003

(51) Int. Cl. A61F 13/15; A61F 13/20

(52) U.S. Cl. 604/387; 604/385.24

(57) ABSTRACT

A diaper type undergarment for a semi-incontinent male is disclosed. The diaper type undergarment includes overlapping absorbent panels that create an opening through which a male wearer may urinate. The opening allows a semi-incontinent user to wear the undergarment and use a urinal in a public restroom without the need for removing pants or the undergarment.

### BACKGROUND OF THE INVENTION

Disposable diapers have been used for many years for infants and incontinent adults. The adult diapers are typically larger sized versions of the infant diapers. Thus, many of the adult diapers include the same features of the smaller sized diapers for infants. Typically, the adult diapers are designed to fit both sexes even though it is well-known that males and females are anatomically different.

After undergoing prostrate or other types of surgery, some males may develop bladder control problems. They may become incontinent or semi-incontinent. Embarrassment associated with bladder control problems may cause an individual to withdraw from society. It is therefore desirable to provide a diaper that is especially designed for semi-incontinent and incontinent males.

Adult diapers are used to counter the effect of embarrassment created by being incontinent. However, use of an adult diaper by males may also lead to embarrassment. For example, men's public restrooms typically include urinals. In order to use a urinal, an adult male wearing a diaper must pull his pants and the diaper down, thereby showing other restroom users that he is wearing a diaper and alerting them that he may be incontinent. This may cause undue stress and embarrassment. Moreover, a semi-incontinent or individual having bladder control problems may not have time to pull down his pants and undergarment when going to a restroom.

None of the adult diapers currently on the market are designed towards semi-incontinent males. Therefore, semi-incontinent adults are treated as if they are completely incontinent and required to wear diapers accordingly. The present invention addresses the problem of one design for all adult diapers. More specifically, the present invention provides a novel design for males that may be incontinent or semi-incontinent.

Although particular embodiments of the invention will be described and illustrated hereinafter, it will be apparent to one skilled in the art that numerous modifications and variations can be implemented without departing from the basic concept. It is to be understood, however, that such modifications and variations will fall within the full scope of the invention as defined by the claims that follow the written description of the invention.

### SUMMARY OF THE INVENTION

The present invention solves the above-mentioned problems for semi-incontinent males. Generally, the invention is directed towards a diaper style undergarment for incontinent and semi-incontinent males. More specifically the invention is directed towards a diaper style undergarment having an opening in the frontal region of the diaper. In the preferred embodiment, the opening is formed by overlapping the front and back panels to create an opening that advantageously allows the user to urinate while keeping the undergarment securely about the user's waist. The sides of the back panel of the undergarment include fastening tabs that allow a caregiver or male wearer to conveniently check and change the undergarment without removing the wearer's clothing. Elastic provided around a leg region and in the inner crotch help prevent leakage. Elastic is also provided around the waist region.

The present invention includes overlapping padded panels that design an opening through which a wearer may readily access and pass his penis in order to urinate. Thus, the undergarment of the present invention allows an adult male to conceal the wearing of the undergarment when using a urinal. The wearer can present himself in public without other individuals knowing that he may have a bladder control problem. While in public and after realizing the urge to urinate, a wearer travels to the restroom. He can then readily use a urinal without dropping his pants and pulling down his undergarment. Moreover, the instant diaper is durable and may be worn for extended periods of time since the wearer does not need to drop his pants to urinate and is therefore less likely to urinate in the undergarment.

In one embodiment, the invention includes an hourglass-shaped diaper-type undergarment that can be assembled about a wearer's waist and pelvic region. The undergarment includes an exterior cloth-like material that is hydrophobic or waterproof. The exterior cloth-like material may comprise a sheet of polyethylene material. Or, it may include a liquid impermeable layer externally covered with a cloth-like material. The undergarment also includes several absorbent pads. Two overlapping pads create a side opening in a front region of the undergarment. Thus, in one embodiment the undergarment is produced by overlapping a front panel and a back panel to create an opening in the front region of the undergarment.

However, it should be noted that other materials may be utilized in implementing the invention. For example, various types of material may be substituted for the material disclosed in this description without deviating from the scope of this invention. It should also be noted that the overlapping panels may be replaced by a single pad having a diagonal slit or opening in the frontal region. The diagonal slit is cut from a left interior side to a right exterior side or visa versa. The invention may be implemented in underpants or pull-up type diapers.

The undergarment may also include an absorbent inner filling and an odor absorbent pad. The absorbent inner filling material may comprise cellulose pulp or a mixture of super absorbent material. It may further include a thin absorbent pad located in the crotch region. Elastic may be affixed about the sides of the crotch region. A thin wall of elasticized moisture repellant material may be included near the exterior edges of the crotch region and extend from the front region to the back region of the undergarment. Hook and latch type material is affixed to an exterior side of the back region for mating with complementary hook and latch type material on the exterior side of the front region. In the alternative, the hook and latch type material may be substituted for an adhesive tape or the like.

In the preferred embodiment, the undergarment includes both a front and back panel. The front panel includes an exterior cloth-like material that is waterproof. A first inner absorbent pad is attached to the exterior cloth-like material and may include odor absorbent properties. An absorbent filling material may be sandwiched between the exterior cloth-like material and the first inner absorbent pad. A second inner absorbent pad is sandwiched against the first inner absorbent pad. Absorbent filling material may be affixed between the first and second absorbent pads. Elastic material may be included at the top of the exterior cloth-like material. An interior moisture permeable cloth-like material covers the pads and any filling material. Soft elastic-type moisture barriers are provided on the interior cloth-like material for preventing any body fluids from leaking from the undergarment. Elastic-like material is provided on the edges of the front panel where the exterior and interior cloth-like materials meet.

The back panel comprises interior and exterior cloth-like materials similar to those of the front panel. Inner absorbent pads are sandwiched between the interior and exterior cloth-like materials, similar to the arrangement of those in the front panel. Likewise elastic is provided on the top of the back panel and along the sides that comprise the crotch region of the finished product. Velcro® tabs or other similar fastening means are included on the exterior cloth-like material near the top of the back panel for mating with complementary strips on the exterior of the front panel.

It is an object of the invention to provide an undergarment that can be conveniently worn to public places.

It is another object of the invention to provide a diaper style undergarment for a semi-incontinent male that can be worn without drawing unnecessary attention to the wearer.

It is an object of the invention to provide an undergarment for a semi-incontinent or incontinent male that alleviates stress and embarrassment relating to an incontinent condition.

### BRIEF DESCRIPTION OF THE DRAWINGS

FIG. 1 is a perspective front view of the undergarment of the present invention.

FIG. 2 is an exterior view of the front panel of the undergarment showing the waist and crotch regions separated.

FIG. 3 is an exterior view of the front panel of the undergarment.

FIG. 4 is an exterior view of the back panel of the undergarment showing the waist and crotch regions separated.

FIG. 5 is an exterior view of the back panel of the undergarment.

FIG. 6 is an exterior view of the undergarment showing the overlapping of the front and back panels of the undergarment.

FIG. 7 is an interior view of the undergarment showing the overlapping of the front and back panels of the undergarment.

FIG. 8 is an interior view of the undergarment having elastic and elasticized moisture barriers.

### DETAILED DESCRIPTION OF THE INVENTION

Although particular embodiments of the invention have been described and illustrated it will be apparent to one skilled in the art that numerous modifications and variations can be implemented without departing from the basic concept. It is to be understood, however, that such modifications and variations will fall within the full scope of the invention as defined by the claims following the subsequent discussion of the invention.

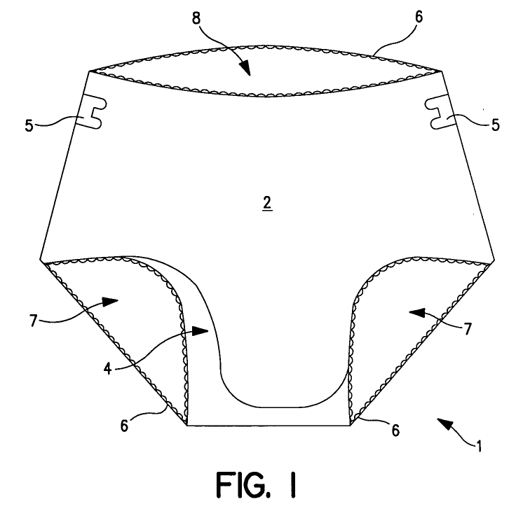

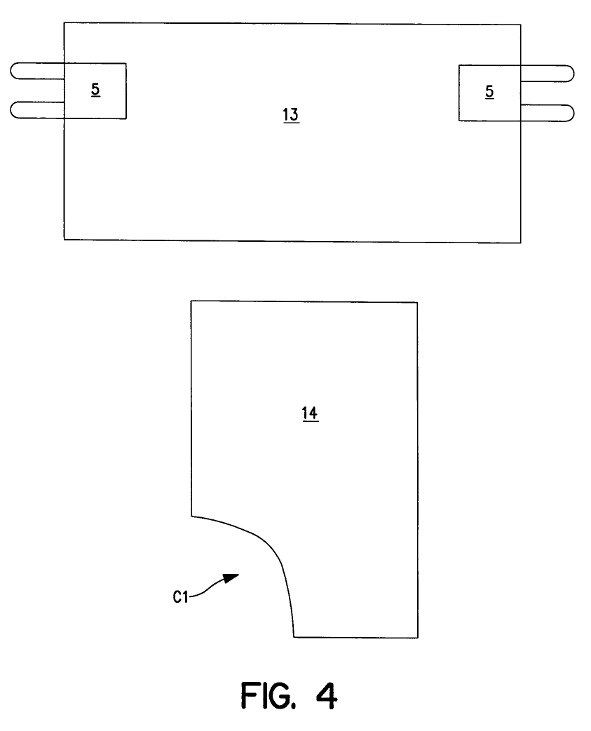

FIG. 1 is a front view of the undergarment 1. The undergarment includes fastening tabs 5 for coupling the front and back waist regions together. Side opening 4 is created by overlapping the front panel 2 and the back panel 3 (shown in FIGS. 4-8). Elastic 6 is affixed around an edge of the waist opening 8 and leg openings 7.

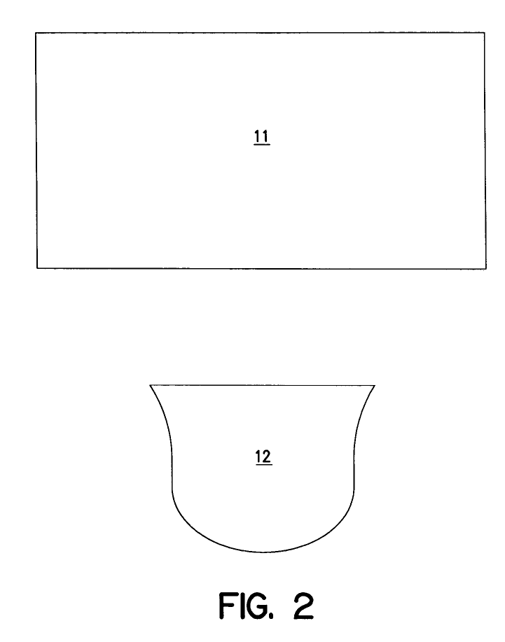

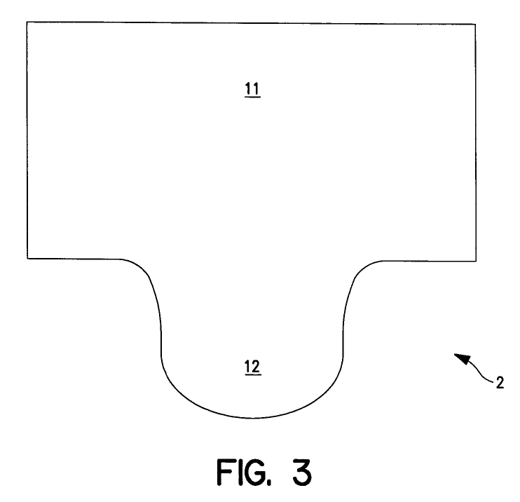

FIGS. 2 and 3 are views showing the crotch region 12 and the waist region 11 of the front panel 2. The crotch region 12 is generally tongue-shaped as shown. However, the edge of the crotch region nearest side opening 4 (FIG. 1) may include a curved shape to help form the side opening 4. The waist region 11 is generally rectangular in shape. The crotch and waist regions may be formed by fusing, bonding, stitching or cutting absorbent materials in this shape. The crotch region 12 and the waist region 11 of the front panel 2 are shown together in FIG. 3.

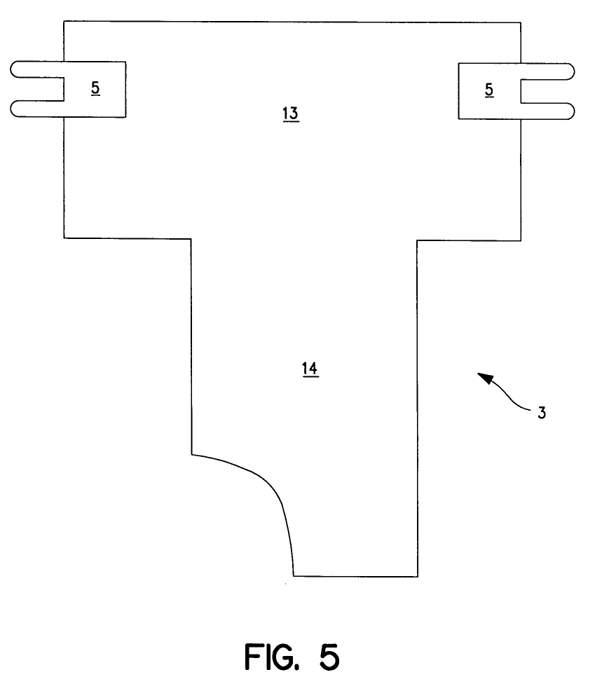

FIGS. 4 and 5 are views showing the back panel 3. Back panel 3 includes a waist region 13 and crotch region 14. Fastening tabs are affixed to waist region 13 for coupling waist region 13 to waist region 11 to allow the user or caregiver to quickly remove and replace the undergarment 1. Crotch region 14 includes a removed section in the shape of arc Cl that helps form side opening 4.

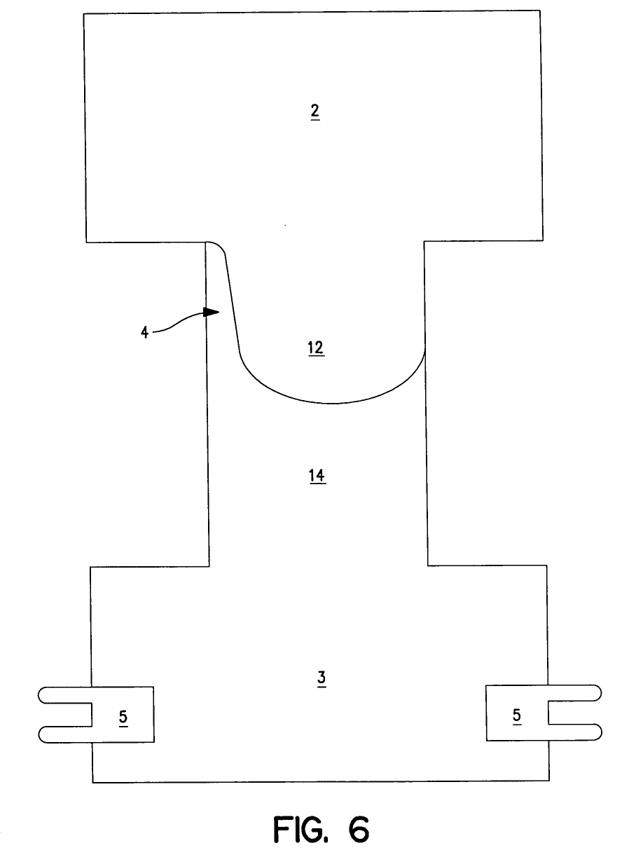

FIG. 6 is an exterior view of the undergarment 1 showing the overlapping of the front panel 2 and the back panel 3. The panels 2 and 3 may be bonded together by using adhesives, sonic welding techniques, sewing techniques or fastening methods. The width of the crotch region 12 is shown as being narrower than the width of the crotch region 14 in this embodiment. However, the invention may be implemented by having the width of both crotch regions being equal and having removed portions in the shape of arcs to create the side opening.

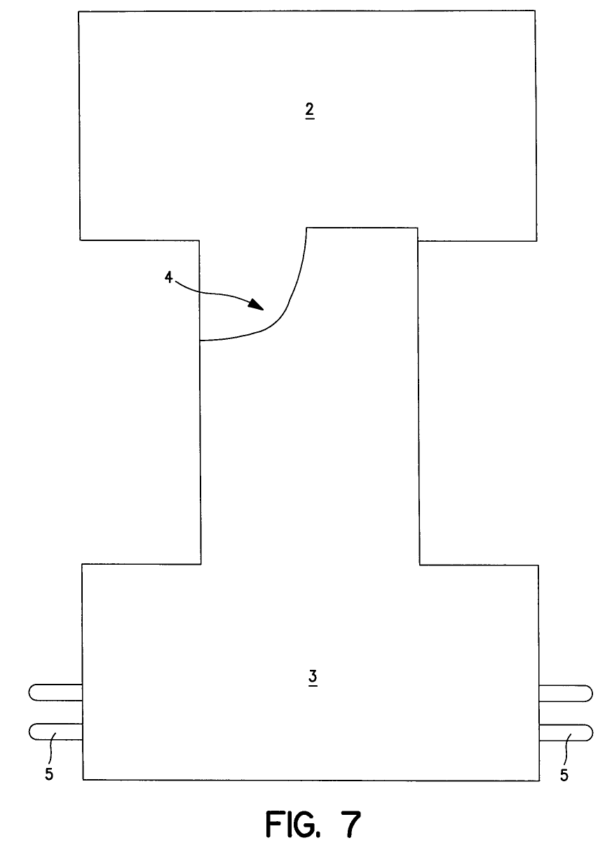

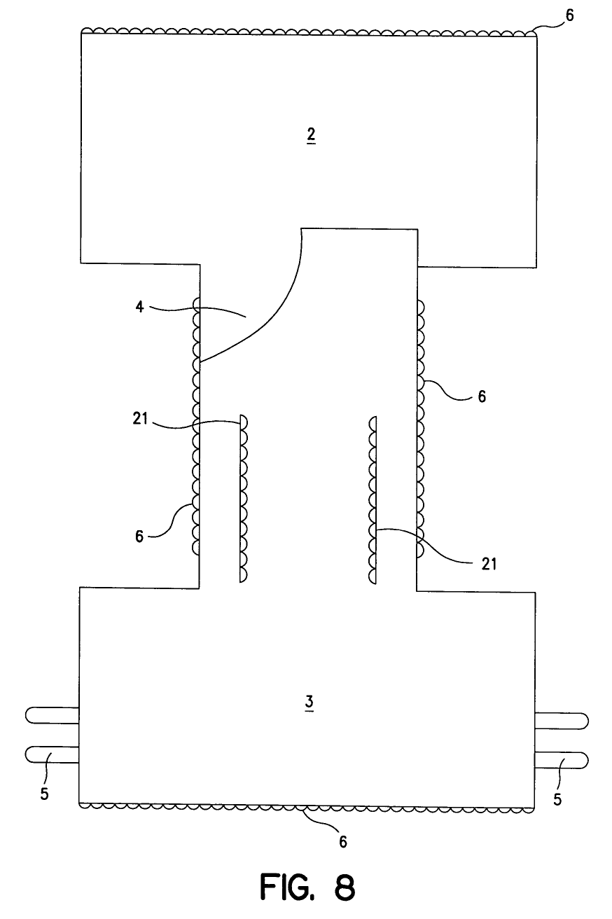

FIG. 7 and 8 are interior views of the undergarment 1 showing the overlapping back panel 3 and the front panel 2. In FIG. 8, the undergarment 1 is equipped with elastic 6 and elasticized moisture barriers 21.

It should be noted that the arrangement of elements may be alternated such that different materials and elements that serve the same function and purpose as those discussed above may be substituted to implement the invention. It is to be understood that the invention is not limited to the exact construction illustrated and described above. Various changes and modifications may be made without departing from the spirit and the scope of the invention as defined in the following claims.

We claim:

1. A diaper for an incontinent or semi-incontinent person comprising:
a first panel of absorbent material including a waist region and a crotch region;
a second panel of absorbent material including a waist region and a crotch region, the crotch region of said second panel overlapping and fastening to at least a portion of the crotch region of said first panel to create an opening that extends from an interior of the diaper to an exterior thereof; and,
two leg openings on opposite sides of said crotch regions.

2. The diaper of claim 1 further comprising at least a first fastening tab affixed to an exterior of the waist region of the second panel; and, a second fastening tab affixed to the exterior of the waist region of the second panel.

3. The diaper of claim 1 further comprising an exterior covering of liquid impervious material.

4. The diaper of claim 1 further comprising an interior covering of liquid absorbent material.

5. The diaper of claim 1 further comprising an elastic material affixed around an edge of the leg openings.

6. The diaper of claim 1 further comprising elasticized materials affixed to a crotch region of the diaper near the leg openings for preventing body fluids from leaking from the leg openings.

7. The diaper of claim 1 further comprising elasticized materials affixed to a waist region of the diaper.

8. An undergarment including an interior covering and an exterior covering, said undergarment comprising:
a front region that includes two overlapping absorbent panels that create an opening that extends from an interior of the undergarment to an exterior of the undergarment;
a crotch region having a front end and a back end and two edges, said front end of the crotch region connecting to said front region; and
a back region connected at an end to said back end of said crotch region.

9. The undergarment of claim 8 further comprising elastic material affixed to an interior of said crotch region for helping prevent body fluids from leaking from the leg openings.

10. The undergarment of claim 8 further comprising:
at least a first fastening tab affixed to an exterior of the second panel; and,
a second fastening tab affixed to the exterior of the second panel on an opposite side of the first fastening tab.

11. The undergarment of claim 8 further comprising an exterior covering of liquid impervious material.

12. The undergarment of claim 8 further comprising an interior covering of liquid absorbent material.

13. The undergarment of claim 12 further comprising elasticized materials affixed to the crotch region of the diaper near the leg openings for preventing body fluids from leaking from the leg openings.

14. The undergarment of claim 8 further comprising an elastic material affixed around an edge of the crotch region.

15. The diaper of claim 1 further comprising elasticized materials affixed to the front and back regions of the diaper.

16. An undergarment having a front and back and comprising:
a first panel that comprises a waist region and substantially tongue-shaped crotch region, said waist region including elastic attached to an edge thereof, at least a portion of said crotch region forming leg openings and elastic attached thereto.

17. The undergarment of claim 16 further comprising elasticized materials affixed to said crotch region of said second panel for preventing body fluids from leaking from the leg openings.
